(19) United States
(12) Patent Application Publication
(10) Pub. No.: US 2026/0072002 A1
(43) Pub. Date: Mar. 12, 2026

(54) SYSTEMS AND METHODS FOR DYNAMIC IDENTIFICATION AND REPORTING OF ADULTERANTS IN HONEY

(71) Applicant: University of Scranton, Scranton, PA (US)

(72) Inventor: Gerard DUMANCAS, Scranton, PA (US)

(73) Assignee: University of Scranton, Scranton, PA (US)

(21) Appl. No.: 18/826,943

(22) Filed: Sep. 6, 2024

(51) Int. Cl. G01N 33/02; G01N 21/35; G01N 21/3577; G01N 21/552

(52) U.S. Cl. CPC G01N 33/02; G01N 21/3577; G01N 21/552; G01N 2021/3595

(57) ABSTRACT

A smartphone application and mid-infrared spectrometric system for the analysis of honey. The system enables the determination of syrup and sugar adulterants, moisture content, and floral and geographical origin of honey using advanced machine learning and chemometric techniques. The state-of-the-art system may revolutionize honey purity testing by determining the quantity of the aforementioned parameters (e.g., quantity of syrup and sugar adulterants, percentage of moisture content, and floral and geographical origin of honey) in only 1-2 minutes. The system can be used in situ, thus offering the advantages of portability and convenience.

### CROSS REFERENCE TO RELATED APPLICATIONS

This application claims the priority and benefit to U.S. Provisional Application No. 63/596,466 filed on Nov. 6, 2023, which is incorporated in its entirety as a part hereof for all intents and purposes.

### TECHNICAL FIELD

The present disclosure relates generally to the problem of adulterated foods and, more particularly, to detecting the purity or lack of purity (e.g., adulteration) of honey.

### BACKGROUND OF THE INVENTION

Honey is a thick, sugary, concentrated nectar (18% water) that consists primarily of fructose and glucose and minor components such as sucrose, proteins, enzymes, amino and organic acids, lipids, vitamins, volatile chemicals, phenolic acids, flavonoids, and minerals. Due in part to its sweet taste and sticky texture, honey is one of the most widely used sweeteners in the food industry and has become a staple ingredient in many kitchens. Aside from its culinary applications, honey also has several medical applications, e.g., it has both anti-aging and anti-bacterial properties and can also be used as a remedy for sore throat. As the human population grows, the consumption of honey has increased worldwide.

The growing demand for honey has rendered adulteration (which is the addition of any substance to the pure honey) commensurately more common. Honey is now the third most adulterated product in the world. Adulteration may be done directly by adding adulterants to the honey itself or indirectly by overfeeding sugar sirups to bee colonies as supplements during periods of low honey production. Adulterants most frequently added to honey include syrups of corn, cane, beet, and rice, which have economic and organoleptic consequences. According to the American Honey Producers Association, much of the Chinese honey that continues to be illegally imported into the United States through third-world countries has been blended with hard-to-detect, low-value sugars like rice syrup. The U.S. Customs and Border Protection lacks both the equipment and knowledge necessary to detect such illegal imports and has not made such detection an agency priority. In fact, honey safety and quality are not regularly monitored and there is currently no U.S. federal standard for the identification of pure honey, which hampers regulatory efforts to ensure the product's quality and safety.

Adulteration not only lowers the quality of honey; adulterated honey poses risks to consumer health such as higher blood sugar, increased risk of diabetes, and weight gain. The decline in honey quality also lowers the perceived level of confidence that consumers have in the nutritional value this product offers, making the task of marketing honey to the public more challenging. Honey evaluation would therefore not only help to improve the quality of honey and minimize the risks of adulteration but would also assist in improving the appeal of honey to the public.

Although honey sugar compositions are complex, honey sugar profiles can be used as a fingerprint to verify the authenticity or determine the adulteration of honey by the addition of sirups and other sweeteners. Current methods to analyze honey adulteration use third-party laboratories outside the United States, such as Intertek and Eurofins in Germany. Honey samples are shipped from the United States to Germany and the results typically take 1-2 weeks to arrive. The average Intertek price of their service starts from $800 per sample. A less expensive and more timely option would be an improvement.

Previous methods have assessed the presence of syrup adulterants in honey but are primarily focused on improving the discrimination of adulterated honey from authentic honey. Further, most methods have focused on the rapid detection of sugar adulterants (e.g., D-fructose, D-glucose, etc.) or on a few syrup honey adulterants (i.e., one or two adulterant matrices), and in many cases use bulky instrumentation such as a nuclear magnetic resonance (NMR) spectrometer, a high performance liquid chromatograph (HPLC), a stable isotope ratio mass spectrometer, or a gas chromatograph-mass spectrometer (GC-MS). Methods using these instruments are considered to be complex, tedious, time consuming, and expensive. Operators must be skilled in handling these instruments; for example, the NMR spectrometer risks a lack of resolution leading to signal overlapping and the need for an “expert interpretation.” Simply put, agents with the U.S. Customs and Border Protection are unlikely to know how to use or have access to complex analytical instrumentation. The use of thin layer chromatography is another method proposed for honey adulteration detection. Although thin layer chromatography has the advantages of simplicity and speediness, extensive work is still needed to assess its reliability. Again, agents with the U.S. Customs and Border Protection are unlikely to know or have access to such analytical instrumentation.

Thus, rapid, facile, and accurate detection and characterization of honey adulteration still remains a need and a continuing challenge to the security of honey.

### BRIEF SUMMARY OF THE INVENTION

To meet these and other needs and challenges, and in view of its purposes, the present disclosure provides a smartphone application and mid-infrared spectrometric system for the direct determination of syrup and sugar adulterants, moisture content, and floral and geographical origin of honey using advanced machine learning and chemometric algorithms. The system is portable, convenient, and easy-to-use. Included in the system is a mid-infrared micro-Fourier transform infrared (FTIR) spectrometer configured to apply a spectrometric analysis to a honey sample. An attenuated total reflectance (ATR) accessory is located in the spectrometer and configured to receive the honey sample and operate on the sample by measuring the changes that occur in an internally reflected infrared beam when the beam comes in contact with the sample. A microcomputer is located in the spectrometer and configured to apply chemometrics to the system. A smartphone is connected to the microcomputer located in the spectrometer either via a connecting cord or via a wireless communication signal, the smartphone having a display that shows a user the results of the analyses completed by the spectrometer, the accessory, and the microcomputer and a keypad that gives the user the ability to control the operations of the system.

The disclosed system includes a server comprising a processor aided by memory that communicates with sensor(s) and database(s). The database(s) contains: (1) a plurality of predetermined individual chemometric measurements, which indicate adulterants, (2) a plurality of predetermined individual chemometric measurements, which indicate moisture content, (3) a plurality of predetermined individual chemometric measurements which indicate origin of the food product, and (4) at least one safety threshold, and linked ranges of tolerance, related to the adulteration, moisture content, and origin of a food product, the predetermined individual chemometric measurements, threshold and range of tolerance having been previously uploaded by an individual or agency administering the test or an employee, contractor, or agent of the individual or agency.

The disclosed method includes receiving information related to a sample of the food product from a sensor for monitoring the food product and optionally testing environment synced through a wired and/or wireless communication network with a software application operating on a mobile computer device or on a computer device. Upon receiving the information, using a processor to determine an individual product profile and call up: (1) a plurality of predetermined individual chemometric measurements, which indicate adulterants, (2) a plurality of predetermined individual chemometric measurements, which indicate moisture content, (3) a plurality of predetermined individual chemometric measurements which indicate origin of the food product, and (4) at least one safety threshold, and a linked range of tolerance, related to the adulteration, moisture content, and origin of a food product, the predetermined individual chemometric measurements, threshold and range of tolerance having been previously uploaded by an individual or agency administering the test or an employee, contractor, or agent of the individual or agency. Next, creating an individual risk profile by comparing the information received from the sensor against the predetermined individual chemometric measurements, which indicate adulterants, moisture content, or origin of the food product. Then, comparing the individual risk profile to the at least one safety threshold, and a linked range of tolerance. As a result, the method notifies the software application if the individual risk profile is above or below a specified safety threshold; and, optionally, assigns additional resources when the security profile is above the specified threshold risk level.

It is to be understood that both the foregoing general description and the following detailed description are exemplary, but are not restrictive, of the disclosure.

### BRIEF DESCRIPTION OF THE DRAWING

The disclosure is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1 is a schematic diagram of a system including a smartphone application with a micro-Fourier transform infrared (FTIR) spectrometer device according to the present disclosure;

FIG. 2 shows one example of a microcomputer that can be included in the FTIR spectrometer device illustrated in FIG. 1; and

FIG. 3 illustrates an example computer system that can be used as part of or in connection with the system according to the present disclosure.

FIG. 4 illustrates an exemplary embodiment of the disclosed system and method for adulteration detection and classification.

### DETAILED DESCRIPTION OF THE INVENTION

The invention provides solutions to the present need in the art for systems and methods for the rapid, facile, and accurate detection and characterization of honey adulteration. The invention solves the prior art problems using a computer-based test platform that is specially programmed to identify adulterants and/or the provenance of a liquid confectionary sample and report out the same to relevant oversight managers (e.g., notify FDA employees if an adulterant is detected). The invention can include a computerized test administration system that functions with a reporting management system in order to allocate food security resources intelligently. Various embodiments of the invention are described in detail below.

In an embodiment, the system analyzes a sample to assess purity and provenance associated with the sample. The data collection and use of that information for continuous assessment of food security has never been performed in real-time. As outlined above, prior art systems collect little information, are cumbersome and time consuming, which allows adulterated food to enter the marketplace without proper vetting (e.g., whether or not to interdict a delivery before it is offered to consumers because of an adulterant; confirm the claimed product origin is in fact true; etc.).

The invention provides for identifying risk levels of adulterants and mis-marking of product origins so that security resources can be applied intelligently and dynamically. Interdiction resources are best applied to high-risk products. For example, the detection of an adulterant in a sample (i.e., the product has to be pulled from the shelf or packaging shut down) should be allocated greater security resources than a low-risk or no-risk circumstance, such as determining the type of wildflower honey (e.g., dandelion). Indeed, low-risk events may only be allocated automated security monitoring resources as opposed to personal notification of security resources. This dynamic resource allocation is an improvement over current resource management methods, which currently results in using the same resources regardless of whether the security risk is low or high. This type of dynamic resource allocation provided by the invention has not been performed and can significantly reduce the expense to government agencies and food packers. The drastically lower costs of such test administration and response encourages testing of the food supply anywhere at any time. This embodiment of the invention provides security for high-risk events, more accessibility to testing, and value to areas of the world where testing has been economically hindered.

With dynamic resource allocation provided by this invention, an oversight manager (e.g., an employee/agent of the FDA or food packer) can analyze and clear a food product for entry into the US food system at any location and at any time. This flexibility to test food products anywhere and anytime provides significant benefits for food safety and consumers. For example, testing can occur in places where the food is packaged.

A detailed discussion of the methods and systems of the invention is provided below. First, a system overview is discussed. Next, a step-by-step approach to dynamic food security is outlined. Third, the device(s) that the system employs are identified. A description of a cloud computing system follows. The advantages provided by the disclosed system(s)/method(s) are described. Finally, incorporation of additional parameters are discussed.

### System Overview

A system for detecting adulteration, moisture content, and origin of a food product, the system comprising:
a software application operating on a mobile computer device or on a computer device, which are in communication with at least one sensor for producing a spectral image of a sample of the food product, the software application configured to receive information related to chemometrics of the sample from the sensor and to communicate the information through a wired and/or wireless communication network to an oversight management server located at the test location or at a location remote from the test location, and
a processor that is in communication through the wired and/or wireless communication network with the software application, as well as the oversight management server, the processer is configured to call up from a database of the system upon communication of the information to the oversight management server: (1) a plurality of predetermined individual chemometric measurements, which indicate adulterants, (2) a plurality of predetermined individual chemometric measurements, which indicate moisture content, (3) a plurality of predetermined individual chemometric measurements which indicate origin of the food product, and (4) at least one safety threshold, and a linked range of tolerance, related to the adulteration, moisture content, and origin of a food product, the predetermined individual chemometric measurements, threshold and range of tolerance having been previously uploaded by an individual or agency administering the test or an employee, contractor, or agent of the individual or agency;
whereby the processor is configured to actively monitor the information related to chemometrics of the sample;
whereby the processor is configured to determine an individual product profile by comparing the information received from the sensor against the individual chemometric measurements to identify whether there are adulterants present in the sample, the moisture content of the sample, and the origin of the sample;
whereby the processor is configured to determine a security profile for the sample by comparing the individual product profile to the safety threshold;
whereby when the processor determines that the individual product profile is above the threshold within the range of tolerance, the processor is configured to notify the software application; and
whereby the software application notifies the individual or agency administering the test or the employee, contractor, or agent of the individual or agency.

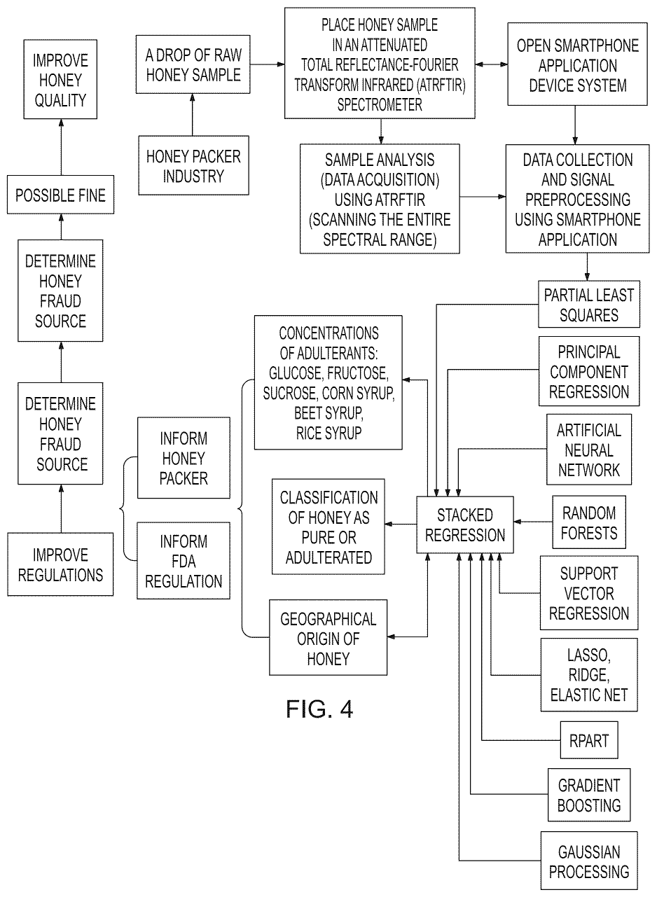

FIG. 4 illustrates the system that includes a server comprising a processor aided by memory that communicates with sensor(s) and database(s). The database(s) contains: (1) a plurality of predetermined individual chemometric measurements, which indicate adulterants, (2) a plurality of predetermined individual chemometric measurements, which indicate moisture content, (3) a plurality of predetermined individual chemometric measurements which indicate origin of the food product, and (4) at least one safety threshold, and a linked range of tolerance, related to the adulteration, moisture content, and origin of a food product, the predetermined individual chemometric measurements, threshold and range of tolerance having been previously uploaded by an individual or agency administering the test or an employee, contractor, or agent of the individual or agency.

In certain embodiments, the system comprises two or three safety thresholds, and linked ranges of tolerances. Such thresholds and ranges of tolerance may be related to: (a) adulterants, (b) moisture contents, (c) origin of the food product, or (d) a combination thereof. Furthermore, the predetermined individual chemometric measurements may be spectral images.

The system may operate on both a software application located on a mobile computer (e.g., mobile phone, tablet, phablet, etc.) or on a website accessible by the mobile computer device.

### Process Flow

As outlined above, the invention relates to a method and system for detecting and classifying adulteration in honey samples. The process involves using an attenuated total reflectance-Fourier transform infrared (ATR-FTIR) spectrometer integrated with a smartphone application to analyze honey samples. Data acquisition, preprocessing, and analysis are conducted using advanced machine learning techniques, including Partial Least Squares (PLS), Principal Component Regression (PCR), Artificial Neural Networks (ANN), and various regression models. The system informs relevant stakeholders, including honey packers and regulatory bodies, and offers capabilities to trace the geographical origin of honey and improve industry regulations.

The system comprises an ATR-FTIR spectrometer, a smartphone application, and a cloud-based machine learning platform. The ATR-FTIR spectrometer is used to scan the honey samples, capturing the entire spectral range necessary for adulteration detection. The smartphone application interfaces with the spectrometer for data collection, preprocessing, and initial analysis. Machine learning models are employed on the cloud-based platform to analyze the preprocessed data and classify the honey samples.

The disclosed system and methods have at least 5 steps. First sample preparation and spectral data acquisition occurs. In this step, a raw honey sample is placed in the ATR-FTIR spectrometer, and the spectrometer scans the sample across the full spectral range to capture detailed information about the sample's composition. Second, data collection and signal preprocessing occur. In this step, spectral data is acquired from the ATR-FTIR and transmitted to a processor that preprocesses the raw data, including noise reduction, baseline correction, and normalization to ensure accuracy in subsequent analysis. Third, data analysis using machine learning models occurs. In this step, the preprocessed data is analyzed using various machine learning models previously uploaded by a stakeholder or an employee/agent of the stakeholder. Such models may include: (1) Partial Least Squares (PLS) which may be utilized for predicting concentrations of known adulterants like glucose, fructose, sucrose, and various syrups; (2) Principal Component Regression (PCR) which may be utilized for dimensionality reduction and modeling; (3) Artificial Neural Networks (ANN) which may be utilized for complex pattern recognition and prediction; (4) Random Forests, Support Vector Regression, LASSO, Ridge, Elastic Net, RPART, Gradient Boosting, Gaussian Processing, and Stacked Regression which may individually or in combination be used to enhance the prediction accuracy and reliability of the analysis; or (5) combinations thereof. The processor also calls up a list of known adulterants from a spectral database which may be located at the site of the stakeholder or a site remote from the stakeholder. Using the list and model(s) the processor determines the presence and concentration of potential adulterants and classify the honey as either pure or adulterated.

The final analysis results are communicated to the relevant stakeholders or their employees and/or agents. Such stakeholders may be (1) honey packers and the communication may inform them about the purity or adulteration status of the honey batch; (2) regulatory bodies (e.g., the FDA) who may receive notifications if the honey is adulterated, thus, aiding in regulatory enforcement and potential actions; or (3) both so as to alert regulatory authorities and prevent the adulterated product from being packaged and offered to customers.

Furthermore, the spectral database may include spectral data, previously uploaded to the spectral database by a stakeholder or an agent/employee of the stakeholder that identifies the geographic origin of the product (e.g., confirming that the honey is authentic Manuka honey). In such cases the disclosed system and methods can be used to trace the origin of the honey, which assists in identifying sources of adulteration.

The information provided by the system aids in improving industry regulations and standards, ensuring higher quality honey products in the market. The system can also help in pinpointing sources of honey fraud, leading to better enforcement and potential fines for non-compliance.

### Devices

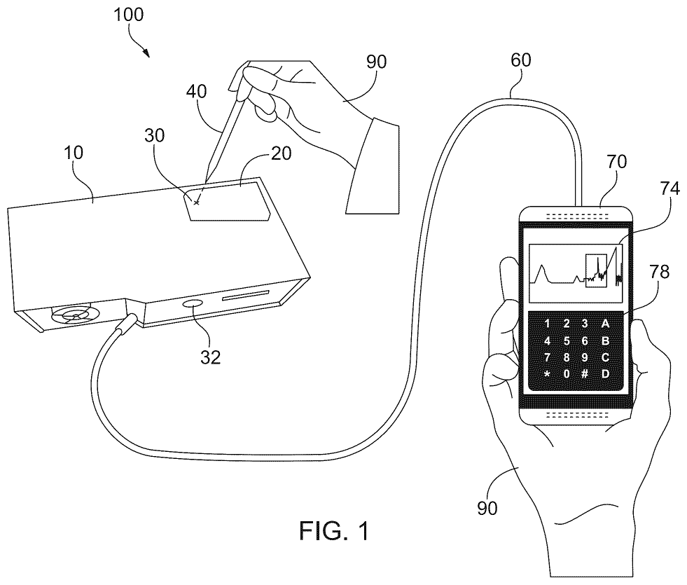

Referring now to the drawing, in which like reference numbers refer to like elements throughout the various figures that comprise the drawing, FIG. 1 is a schematic diagram of a system 100 including a portable computer with a micro-Fourier transform infrared (FTIR) spectrometer device 10 according to the present disclosure. The FTIR spectrometer device 10 is light in weight and small in size and, therefore, is portable. In other words, a user 90 can easily pick up and transport the FTIR spectrometer device 10. In one example, the FTIR spectrometer device 10 weighs about 1 pound and has rectangular dimensions of 2 inches by 2 inches by 2 inches. The FTIR spectrometer device 10 may be contained in a suitable case or housing known in the art, which may include a weatherproof or waterproof box. The specific parts of the FTIR spectrometer device 10 already exist. Thus, a discussion of the principles, functions, and specific details of the FTIR spectrometer device 10 is beyond the scope of this document.

The FTIR spectrometer device 10 includes an attenuated total reflectance (ATR) accessory 20. For information about methods that combine FTIR and ATR, see Catherine Setijadi, Jonathan Felix, Helena Ellis, Jihan Alumbro, Ghalib Bello, and Gerard Dumancas, “Development of a Facile and Convenient Method for Sugar Determination in Low Moisture Confectioneries and Honeys Using Fourier Transform Infrared Attenuated Total Reflectance Spectroscopy and Chemometrics,” Analytical Letters (Taylor and Francis, 2020); and Gerard Dumancas and Helena Ellis, “Comprehensive Examination and Comparison of Machine Learning Techniques for the Quantitative Determination of Adulterants in Honey using Fourier Infrared Spectroscopy with Attenuated Total Reflectance Accessory,” Spectrochimica Acta Part A: Molecular and Biomolecular Spectroscopy 276 (Elsevier B.V., 2022). Both of these publications are incorporated in this document by reference in their entireties.

The ATR accessory 20 is configured to receive a sample 30. The sample 30 is typically a drop of liquid honey. The sample 30 that is required by the FTIR spectrometer device 10 to complete analysis of the sample 30 is very small, on the order of one nanogram to one microgram. The user 90 can deliver a sample 30 from a source of honey under investigation to the ATR accessory 20 using any conventional tool 40, such as a syringe or a dropper. The ATR accessory 20 operates on the sample 30 by measuring the changes that occur in an internally reflected infrared beam when the beam comes in contact with the sample 30.

The FTIR spectrometer device 10 also includes a microcomputer 32 located inside the FTIR spectrometer device 10. The microcomputer 32 is configured to apply chemometrics to the system 100. Thus, the microcomputer 32 can be called a chemometric multivariate calibration chip. Chemometrics is the science of extracting information from chemical systems by data-driven mechanisms. Chemometrics is inherently interdisciplinary, using methods frequently employed in core data-analytic disciplines such as multivariate statistics, applied mathematics, and computer science, in order to address problems.

The use of the mid-infrared region (4,000 to 400 cm⁻¹) associated with chemometrics has shown to be an accurate and fast method to detect and predict food adulteration. For chemometrics, the use of partial least squares (PLS) regression is of particular interest because, unlike multiple linear regression, it has the ability to analyze data which are noisy, redundant, and strongly correlated. Principal component analysis (PCA) is another chemometric technique that is used extensively for screening, extracting, and compressing multivariate data. The main goal of these chemometric methods in conjunction with spectroscopic techniques is to develop an inexpensive, less time consuming, and easy to use application method to measure a property of interest in new and unknown samples. See, e.g., Gerard Dumancas, Helena Ellis, Jossie Neumann, and Khalil Smith, “Comparison of Various Signal Processing Techniques and Spectral Regions for the Direct Determination of Syrup Adulterants in Honey using Fourier Transform Infrared Spectroscopy and Chemometrics,” Chemosensors, 2022, 10, 51 (this publication is incorporated in this document by reference in its entirety).

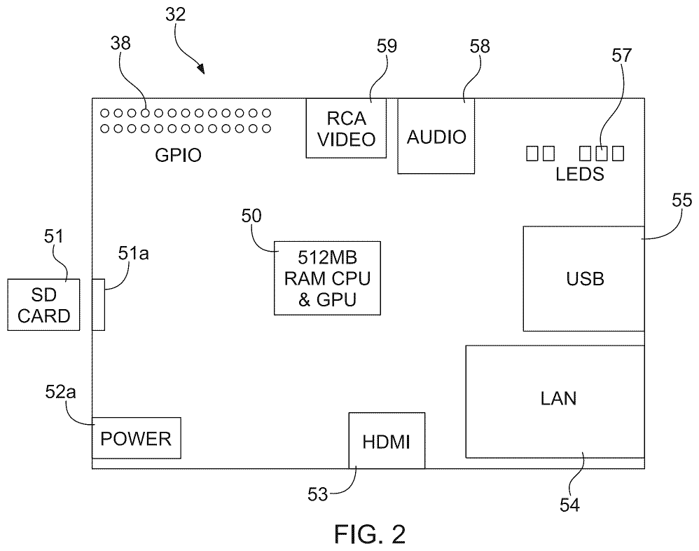

FIG. 2 shows one example of the microcomputer 32 that can be included in the FTIR spectrometer device 10. The microcomputer 32 may be a compact or small microcomputer 32, such as a RASPBERRY PI® microcomputer 32, which can be obtained from the Raspberry Pi Foundation, a UK registered charity, with a website at http://www.raspberrypi.org/. The RASPBERRY PI® microcomputer 32 is a very small credit-card sized microcomputer 32, which is affordable and low cost. For example, as depicted in FIG. 2, the RASPBERRY PI® microcomputer 32 may include a programmable microprocessor with general purpose input and output pins 38 (GPIOs), a single board 50 containing a memory card 51 including the slot 51a for the memory card 51 (e.g., a secure digital (SD) card), a power port 52a, an audio/video interface 53 (e.g., a high-definition multimedia interface (HDMI)), a local area network (LAN) 54 or wireless local area network (WLAN), a universal serial bus (USB) port 55, a combination random access memory (RAM), central processing unit (CPU) and graphics processing unit (GPU) 56 (e.g., a 512 MB RAM CPU and GPU), one or more light displays 57 (e.g., light emitting diodes (LEDs)), an audio output 58, and a video output 59 (e.g., an RCA video output).

The microcomputer 32 is preferably a microcomputer or mini PC comprising a processor or microprocessor. Other mini PCs include the Mac Mini, available from Apple Inc. of Cupertino, California; and any of various Mini Android PCs available from different manufacturers. The processor may execute instructions, codes, computer programs, scripts which it accesses from hard disk, floppy disk, optical disk (these various disk-based systems may all be considered secondary storage), read only memory (ROM), RAM, or network connectivity devices. A microcontroller may not provide the same functionality, however, such as the ability for a user to interface and connect with the system 100 (e.g., to upload and/or retrieve data).

The present disclosure can further be embodied in the form of computer-implemented processes and apparatus for practicing such processes, for example, and can be embodied in the form of computer program code embodied in tangible media, such as floppy diskettes, fixed (hard) drives, CD ROM's, magnetic tape, fixed/integrated circuit devices, or any other computer-readable storage medium, such that when the computer program code is loaded into and executed by the microcomputer 32, the microcomputer 32 becomes an apparatus for practicing the processes. The system 100 includes a calibrated chemometric model derived via FTIR spectrometric experiments. Computational codes will be written using the R statistical programming software. As soon as the processes are developed, such codes will be embedded via the microcomputer 32.

In addition to the FTIR spectrometer device 10, the system 100 also includes a smartphone 70. The smartphone 70 is connected to the FTIR spectrometer device 10 and, more specifically and directly to the microcomputer 32 located in the FTIR spectrometer device 10, either via a connecting cord 60 or via a wireless communication signal. The display 74 of the smartphone 70 shows the user 90 the results of the analysis completed by the FTIR spectrometer device 10 on the sample 30. For example, the display 74 may answer the questions: Is the sample honey pure or not; does the sample contain antibiotics; what is the pollen source; and what is the geographical origin of the sample honey? The display 74 also may identify the composition of the sample honey, giving the percentages of glucose, fructose, sucrose, rice syrup, corn syrup, cane syrup, and beet syrup. The keypad 78 of the smartphone 70 gives the user 90 the ability to control the operations of the smartphone 70 and, in turn, of the FTIR spectrometer device 10.

The calibrated chemometric software model coupled with the FTIR spectrometer device 10 of the system 100 will provide users 90 with a portable, convenient, and easy-to-use instrument for detecting honey adulteration, moisture content, as well as its floral origin. In summary, the system 100 and the overall process implemented using the system 100 will provide a significant potential for a cost and time effective analysis of honey adulteration compared to that of existing methods which use HPLC, NMR, carbon isotope ratio, and GC-MS devices. The system 100 allows quantification of syrup adulterants (corn syrup, cane syrup, beet syrup, and rice syrup) and sugar adulterants (glucose, fructose, sucrose) in honey, and determines the floral origin of honey, in only 1-2 minutes. The system 100 will be configurable for specific sampling applications. Thus, honey analysis will be made easy and convenient.

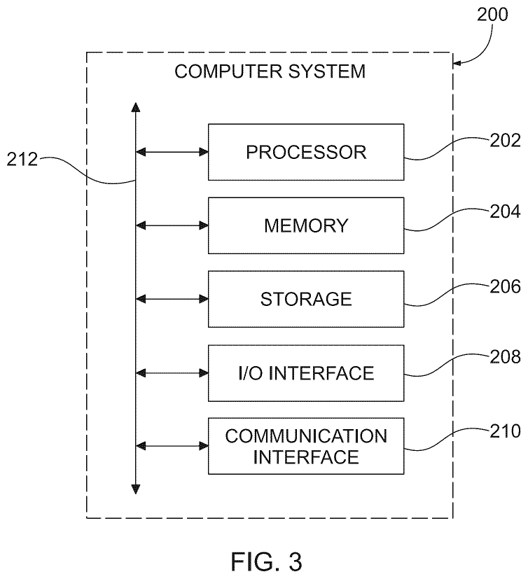

FIG. 3 illustrates an example computer system 200 that can be used either as part of or in connection with the system 100. In other words, the computer system 200 can be used to control the various components (e.g., the FTIR spectrometer device 10, the ATR accessory 20, and the microcomputer 32—and the subcomponents and functionality of these various components) that combine to form the system 100.

In particular embodiments, one or more computer systems 200 perform one or more steps of one or more embodiments of the process implemented on the system 100 described or illustrated in this document. In particular embodiments, one or more computer systems 200 provide functionality described or illustrated in this document. In particular embodiments, software running on one or more computer systems 200 performs one or more steps of one or more embodiments of the process implemented on the system 100 described or illustrated in this document or provides functionality described or illustrated in this document. Particular embodiments include one or more portions of one or more computer systems 200. In this document, reference to a computer system may encompass a computing device, and vice versa, where appropriate. Moreover, reference to a computer system may encompass one or more computer systems, where appropriate.

This disclosure contemplates any suitable number of computer systems 200. This disclosure contemplates the computer system 200 taking any suitable physical form. As example and not by way of limitation, the computer system 200 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these devices. Where appropriate, the computer system 200 may include one or more computer systems 200; be unitary or distributed; span multiple locations; span multiple machines; span multiple data centers; or reside in a cloud, which may include one or more cloud components in one or more networks. Where appropriate, one or more computer systems 200 may perform without substantial spatial or temporal limitation one or more steps of one or more embodiments of the process implemented using the system 100 described or illustrated in this document. As an example and not by way of limitation, the one or more computer systems 200 may perform in real time or in batch mode one or more steps of one or more embodiments of the process described or illustrated in this document. The one or more computer systems 200 may perform at different times or at different locations one or more steps of one or more embodiments of the process described or illustrated in this document, where appropriate.

In particular embodiments, the computer system 200 includes a processor 202, memory 204, storage 206, an input/output (I/O) interface 208, a communication interface 210, and a bus 212. Although this disclosure describes and illustrates a particular computer system having a particular number of particular components in a particular arrangement, this disclosure contemplates any suitable computer system having any suitable number of any suitable components in any suitable arrangement.

In particular embodiments, the processor 202 includes hardware for executing instructions, such as those making up a computer program. As an example and not by way of limitation, to execute instructions, the processor 202 may retrieve (or fetch) the instructions from an internal register, an internal cache, the memory 204, or the storage 206; decode and execute them; and then write one or more results to an internal register, an internal cache, the memory 204, or the storage 206. In particular embodiments, the processor 202 may include one or more internal caches for data, instructions, or addresses. This disclosure contemplates the processor 202 including any suitable number of any suitable internal caches, where appropriate. As an example and not by way of limitation, the processor 202 may include one or more instruction caches, one or more data caches, and one or more translation lookaside buffers (TLBs). Instructions in the instruction caches may be copies of instructions in the memory 204 or the storage 206, and the instruction caches may speed up retrieval of those instructions by the processor 202. Data in the data caches may be copies of data in the memory 204 or the storage 206 for instructions executing at the processor 202 to operate on; the results of previous instructions executed at the processor 202 for access by subsequent instructions executing at the processor 202 or for writing to the memory 204 or the storage 206; or other suitable data. The data caches may speed up read or write operations by the processor 202. The TLBs may speed up virtual-address translation for the processor 202. In particular embodiments, the processor 202 may include one or more internal registers for data, instructions, or addresses. This disclosure contemplates the processor 202 including any suitable number of any suitable internal registers, where appropriate. Where appropriate, the processor 202 may include one or more arithmetic logic units (ALUs); be a multi-core processor; or include one or more processors 202. Although this disclosure describes and illustrates a particular processor, this disclosure contemplates any suitable processor.

In particular embodiments, the memory 204 includes main memory for storing instructions for the processor 202 to execute or data for the processor 202 to operate on. As an example and not by way of limitation, the computer system 200 may load instructions from the storage 206 or another source (such as, for example, another computer system 200) to the memory 204. The processor 202 may then load the instructions from the memory 204 to an internal register or internal cache. To execute the instructions, the processor 202 may retrieve the instructions from the internal register or internal cache and decode them. During or after execution of the instructions, the processor 202 may write one or more results (which may be intermediate or final results) to the internal register or internal cache. The processor 202 may then write one or more of those results to the memory 204. In particular embodiments, the processor 202 executes only instructions in one or more internal registers or internal caches or in the memory 204 (as opposed to the storage 206 or elsewhere) and operates only on data in one or more internal registers or internal caches or in the memory 204 (as opposed to the storage 206 or elsewhere). One or more memory buses (which may each include an address bus and a data bus) may couple the processor 202 to the memory 204. The bus 212 may include one or more memory buses, as described below. In particular embodiments, one or more memory management units (MMUs) reside between the processor 202 and the memory 204 and facilitate accesses to the memory 204 requested by the processor 202. In particular embodiments, the memory 204 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. The memory 204 may include one or more memories 204, where appropriate. Although this disclosure describes and illustrates particular memory, this disclosure contemplates any suitable memory.

In particular embodiments, the storage 206 includes mass storage for data or instructions. As an example, and not by way of limitation, the storage 206 may include a hard disk drive (HDD), a floppy disk drive, flash memory, an optical disc, a magneto-optical disc, magnetic tape, or a Universal Serial Bus (USB) drive or a combination of two or more of these. The storage 206 may include removable or non-removable (or fixed) media, where appropriate. The storage 206 may be internal or external to the computer system 200, where appropriate. In particular embodiments, the storage 206 is non-volatile, solid-state memory. In particular embodiments, the storage 206 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates the storage 206 taking any suitable physical form. The storage 206 may include one or more storage control units facilitating communication between the processor 202 and the storage 206, where appropriate. Where appropriate, the storage 206 may include one or more storages 206. Although this disclosure describes and illustrates particular storage, this disclosure contemplates any suitable storage.

In particular embodiments, the I/O interface 208 includes hardware, software, or both, providing one or more interfaces for communication between the computer system 200 and one or more I/O devices. The computer system 200 may include one or more of these I/O devices, where appropriate. One or more of these I/O devices may enable communication between a person and the computer system 200. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 208 for them. Where appropriate, the I/O interface 208 may include one or more device or software drivers enabling the processor 202 to drive one or more of these I/O devices. The I/O interface 208 may include one or more I/O interfaces 208, where appropriate. Although this disclosure describes and illustrates a particular I/O interface, this disclosure contemplates any suitable I/O interface.

In particular embodiments, the communication interface 210 includes hardware, software, or both providing one or more interfaces for communication (such as, for example, packet-based communication) between the computer system 200 and one or more other computer systems 200 or one or more networks. As an example, and not by way of limitation, the communication interface 210 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 210 for it. As an example and not by way of limitation, the computer system 200 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the computer system 200 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. The computer system 200 may include any suitable communication interface 210 for any of these networks, where appropriate. The communication interface 210 may include one or more communication interfaces 210, where appropriate. Although this disclosure describes and illustrates a particular communication interface, this disclosure contemplates any suitable communication interface.

In particular embodiments, the bus 212 includes hardware, software, or both coupling components of the computer system 200 to each other. As an example and not by way of limitation, the bus 212 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. The bus 212 may include one or more buses 212, where appropriate. Although this disclosure describes and illustrates a particular bus, this disclosure contemplates any suitable bus or interconnect.

In this document, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

This disclosure contemplates one or more computer-readable storage media implementing any suitable storage. In particular embodiments, a computer-readable storage medium implements one or more portions of the processor 202 (such as, for example, one or more internal registers or caches), one or more portions of the memory 204, one or more portions of the storage 206, or a combination of these, where appropriate. In particular embodiments, a computer-readable storage medium implements RAM or ROM. In particular embodiments, a computer-readable storage medium implements volatile or persistent memory. In particular embodiments, one or more computer-readable storage media embody software. In this document, reference to software may encompass one or more applications, bytecode, one or more computer programs, one or more executables, one or more instructions, logic, machine code, one or more scripts, or source code, and vice versa, where appropriate. In particular embodiments, software includes one or more application programming interfaces (APIs). This disclosure contemplates any suitable software written or otherwise expressed in any suitable programming language or combination of programming languages. In particular embodiments, software is expressed as source code or object code. In particular embodiments, software is expressed in a higher-level programming language, such as, for example, C, Perl, or a suitable extension thereof. In particular embodiments, software is expressed in a lower-level programming language, such as assembly language (or machine code). In particular embodiments, software is expressed in JAVA. In particular embodiments, software is expressed in Hyper Text Markup Language (HTML), Extensible Markup Language (XML), JavaScript Object Notation (JSON) or other suitable markup language.

### Cloud System

Cloud computing is a type of Internet-based computing in which a variety of resources are hosted and/or controlled by an entity and made available by the entity to authorized users via the Internet. A cloud computing system can be configured, wherein a variety of electronic devices can communicate via a network for purposes of exchanging content and other data. The system can be configured for use on a wide variety of network configurations that facilitate the intercommunication of electronic devices. For example, each of the components of a cloud computing system can be implemented in a localized or distributed fashion in a network.

The cloud computing system can be configured to include cloud computing resources (i.e., “the cloud”). The cloud resources can include a variety of hardware and/or software resources, such as cloud servers, cloud databases, cloud storage, cloud networks, cloud applications, cloud platforms, and/or any other cloud-based resources. In some cases, the cloud resources are distributed. For example, cloud storage can include multiple storage devices. In some cases, cloud resources can be distributed across multiple cloud computing systems and/or individual network enabled computing devices. For example, cloud computing resources can communicate with a server, a database, and/or any other network enabled computing device to provide the cloud resources.

In some cases, the cloud resources can be redundant. For example, if cloud computing resources are configured to provide data backup services, multiple copies of the data can be stored such that the data is still available to the user even if a storage resource is offline, busy, or otherwise unavailable to process a request. In another example, if a cloud computing resource is configured to provide software, the software can be available from different cloud servers so that the software can be served from any of the different cloud servers. Algorithms can be applied such that the closest server or the server with the lowest current load is selected to process a given request.

A user interacts with cloud computing resources through user terminals or testing devices connected to a network by direct and/or indirect communication. Cloud computing resources can support connections from a variety of different electronic devices, such as servers, desktop computers, mobile computers, handheld communications devices (e.g., mobile phones, smart phones, tablets), set top boxes, network-enabled hard drives, and/or any other network-enabled computing devices. Furthermore, cloud computing resources can concurrently accept connections from and interact with multiple electronic devices. Interaction with the multiple electronic devices can be prioritized or occur simultaneously.

Cloud computing resources can provide cloud resources through a variety of deployment models, such as public, private, community, hybrid, and/or any other cloud deployment models. In some cases, cloud computing resources can support multiple deployment models. For example, cloud computing resources can provide one set of resources through a public deployment model and another set of resources through a private deployment model.

In some configurations, a user terminal can access cloud computing resources from any location where an Internet connection is available. However, in other cases, cloud computing resources can be configured to restrict access to certain resources such that a resource can only be accessed from certain locations. For example, if a cloud computing resource is configured to provide a resource using a private deployment model, then a cloud computing resource can restrict access to the resource, such as by requiring that a user terminal access the resource from behind a firewall.

Cloud computing resources can provide cloud resources to user terminals through a variety of service models, such as Software as a Service (SaaS), Platforms as a service (PaaS), Infrastructure as a Service (IaaS), and/or any other cloud service models. In some cases, cloud computing resources can provide multiple service models to a user terminal. For example, cloud computing resources can provide both SaaS and IaaS to a user terminal. In some cases, cloud computing resources can provide different service models to different user terminals. For example, cloud computing resources can provide SaaS to one user terminal and PaaS to another user terminal.

In some cases, cloud computing resources can maintain an account database. The account database can store profile information for registered users. The profile information can include resource access rights, such as software the user is permitted to use, maximum storage space, etc. The profile information can also include usage information, such as computing resources consumed, data storage location, security settings, personal configuration settings, etc. In some cases, the account database can reside on a database or server remote to cloud computing resources such as servers or database.

Cloud computing resources can provide a variety of functionalities that requires user interaction. Accordingly, a user interface (UI) can be provided for communicating with cloud computing resources and/or performing tasks associated with the cloud resources. The UI can be accessed via an end user terminal in communication with cloud computing resources. The UI can be configured to operate in a variety of client modes, including a fat client mode, a thin client mode, or a hybrid client mode, depending on the storage and processing capabilities of cloud computing resources and/or the user terminal. Therefore, a UI can be implemented as a standalone application operating at the user terminal in some embodiments. In other embodiments, a web browser-based portal can be used to provide the UI. Any other configuration to access cloud computing resources can also be used in the various embodiments.

### Collection of Data

In some configurations, during the testing described above, a storage device or resource can be used to store relevant data transmitted from the sensor(s) or the stakeholder's device to a database over a wired or wireless communication network. For example, a stakeholder may link a specific source (e.g., farm) with a specific spectral profile to link the origin of the honey. Such data may be used to dynamically adjust origin and adulterant profiles, or the calculations based on those spectral profiles.

The data stored can also be incorporated into the disclosed system and methods to refine the testing experience or evaluate food security. For example, if a location experiences an increase in adulterants in a certain field but not other fields from the same location, a location risk factor can be identified during the test which can result in the allocation of additional resources with relation to the specific location at that site. As a result, the information collected can serve more than one purpose (quality of measurement, better security, identification of environmental risk factors, and others). Those purposes can also be routinely evaluated.

### Advantages Over Prior Art

Whether implemented using the computer system 200 or not, the system 100 offers a number of advantages. Among those advantages are that the system 100 (i) has a computer application interface for honey purity (i.e., syrup adulterant and sugar quantification) and floral origin identification; (ii) can quantify moisture content and floral and geographical origin of honey; (iii) provides an easy to use and affordable method for determining honey purity, moisture content, and floral origin; and (iv) includes complex machine learning regression calibration systems for predicting the amounts of honey adulterants, sugars, as well as the floral origin of honey. The predictive modeling algorithm can predict the amounts of corn, cane, beet, and rice syrups as well as the presence of glucose, fructose, and sucrose in honey.

Additional advantages include ease in getting results to determine the presence of adulterants, moisture content, as well as floral and geographical origin of imported honey. Instead of waiting several weeks to obtain results from another country such as Germany, analyses can be done in-house in a matter of minutes using available equipment. Such analyses can be cost effective for honey commercial industries and producers who can realize cost savings because samples will no longer be shipped to a foreign country.

The portable system 100 can be used within or outside the laboratory (i.e., field testing) for real time detection of the aforementioned honey parameters, which is not the case for currently available technologies. The system 100 offers a rapid analysis time (1-2 minutes) as compared to conventional NMR and HPLC methods (1-3 hours) in part because the FTIR spectrometer device 10 requires little or no sample preparation for spectral acquisition. The system 100 is easy to use, with little or no training required to operate the system 100, as compared to other available methods such as the HPLC and NMR, which require specialized training. The system 100 is sensitive, requires a small-sized sample 30, and is nondestructive (i.e., the sample 30 remains intact during analysis) as compared to conventional methods which subject the sample to degradation. The system 100 avoids unnecessary data analysis because results do not need any further spectral interpretation (i.e., results can readily be read). The system 100 is different from any other products currently used by honey commercial industries and producers via a third-party analytical laboratory. In summary, the system 100 provides a portable, convenient, and easy-to-use apparatus that can accurately and rapidly detect honey adulteration, moisture content, as well as floral origin.

The system 100 will allow honey importers, packers, and retailers, as well as food authentication agencies to quickly track the authenticity of their honey products at each step of the supply chain by quantifying the known adulterant syrups in a facile and convenient manner. This will significantly improve the authenticity and traceability of the product. Further, in situ testing for the presence of honey adulterants as well as floral and geographic fingerprinting will allow various honey market segments to demand the appropriate price for their pure honey products in the global market.

The system 100 offers a competitive advantage over other available products by a significant degree of differentiation. Specifically, the system 100 is more convenient than any other available product and has a lower cost. As compared to an NMR instrument which is expensive (a standard 600 MHz NMR costs about $800,000 and the 900 MHz sells for about $5 million), an FTIR spectrometer (i.e., mid infrared) is considerably less expensive with a cost of only $20,000 to $100,000. The advantages of the system 100 can be expected to have an eight-fold impact on the current state of honey adulteration detection, moisture content, as well as floral and geographical origin determination technologies.

Although illustrated and described above with reference to certain specific embodiments, the present disclosure is nevertheless not intended to be limited to the details shown. Rather, various modifications may be made in the details within the scope and range of equivalents of the claims and without departing from the spirit of the disclosure.

What is claimed is:

1. A system for detecting adulteration, moisture content, and origin of a food product, the system comprising:
a software application operating on a mobile computer device or on a computer device, which are in communication with at least one sensor for producing a spectral image of a sample of the food product, the software application configured to receive information related to chemometrics of the sample from the sensor and to communicate the information through a wired and/or wireless communication network to an oversight management server located at the test location or at a location remote from the test location, and
a processor that is in communication through the wired and/or wireless communication network with the software application, as well as the oversight management server, the processer is configured to call up from a database of the system upon communication of the information to the oversight management server: (1) a plurality of predetermined individual chemometric measurements, which indicate adulterants, (2) a plurality of predetermined individual chemometric measurements, which indicate moisture content, (3) a plurality of predetermined individual chemometric measurements which indicate origin of the food product, and (4) at least one safety threshold, and a linked range of tolerance, related to the adulteration, moisture content, and origin of a food product, the predetermined individual chemometric measurements, threshold and range of tolerance having been previously uploaded by an individual or agency administering the test or an employee, contractor, or agent of the individual or agency;
whereby the processor is configured to actively monitor the information related to chemometrics of the sample;
whereby the processor is configured to determine an individual product profile by comparing the information received from the sensor against the individual chemometric measurements to identify whether there are adulterants present in the sample, the moisture content of the sample, and the origin of the sample;
whereby the processor is configured to determine a security profile for the sample by comparing the individual product profile to the safety threshold;
whereby when the processor determines that the individual product profile is above the threshold within the range of tolerance, the processor is configured to notify the software application; and
whereby the software application notifies the individual or agency administering the test or the employee, contractor, or agent of the individual or agency.

2. The system of claim 1, further comprising at least two safety thresholds, and linked ranges of tolerances.

3. The system of claim 2, wherein at least one safety threshold and linked range of tolerance relates to: (a) adulterants, (b) moisture contents, (c) origin of the food product, or (d) a combination thereof.
The system of claim 3, wherein the system includes at least three safety thresholds and linked ranges of tolerance.

5. The system of claim 1, wherein the food product is honey.

6. The system of claim 1, wherein the sensor comprises
a mid-infrared micro-Fourier transform infrared (FTIR) spectrometer configured to apply a spectrometric analysis to the sample;
an attenuated total reflectance (ATR) accessory located in the spectrometer and configured to receive the sample and operate on the sample by measuring the changes that occur in an internally reflected infrared beam when the beam comes in contact with the sample;
a microcomputer located in the spectrometer and configured to apply chemometrics to the system.

7. The system of claim 1, wherein the predetermined individual chemometric measurements are spectral images.

8. A system for detecting adulteration, moisture content, and origin of a food product, the system comprising:
a software application operating on a website accessible through a wired or wireless communications network by a unique mobile computer device or a unique computer device which are synced with at least one sensor for producing a spectral image of a sample of the food product, the software application configured to receive information related to chemometrics of the sample from the sensor and to communicate the information through a wired and/or wireless communication network to an oversight server located at the test location or at a location remote from the test location, and
a processor that is in communication through the wired and/or wireless communication network with the software application, as well as the oversight server, the processer is configured to call up from a database of the system upon communication of the information to the oversight server: (1) a plurality of predetermined individual chemometric measurements, which indicate adulterants, (2) a plurality of predetermined individual chemometric measurements, which indicate moisture content, (3) a plurality of predetermined individual chemometric measurements which indicate origin of the food product, and (4) at least one safety threshold, and a linked range of tolerance, related to the adulteration, moisture content, and origin of a food product, the predetermined individual chemometric measurements, threshold and range of tolerance having been previously uploaded by an individual or agency administering the test or an employee, contractor, or agent of the individual or agency;
whereby the processor is configured to actively monitor the information related to chemometrics of the sample;
whereby the processor is configured to determine an individual product profile by comparing the information received from the sensor against the individual chemometric measurements to identify whether there are adulterants present in the sample, the moisture content of the sample, and the origin of the sample;
whereby the processor is configured to determine a security profile for the sample by comparing the individual product profile to the safety threshold;
whereby when the processor determines that the individual product profile is above the threshold within the range of tolerance, the processor is configured to notify the software application; and
whereby the software application notifies the individual or agency administering the test or the employee, contractor, or agent of the individual or agency.

9. The system of claim 8, further comprising at least two safety thresholds, and linked ranges of tolerances.

10. The system of claim 9, wherein at least one safety threshold and linked range of tolerance relates to: (a) adulterants, (b) moisture contents, (c) origin of the food product, or (d) a combination thereof.

11. The system of claim 10, wherein the system includes at least three safety thresholds and linked ranges of tolerance.

12. The system of claim 8, wherein the food product is honey.

13. The system of claim 8, wherein the sensor comprises
a mid-infrared micro-Fourier transform infrared (FTIR) spectrometer configured to apply a spectrometric analysis to the sample;
an attenuated total reflectance (ATR) accessory located in the spectrometer and configured to receive the sample and operate on the sample by measuring the changes that occur in an internally reflected infrared beam when the beam comes in contact with the sample;
a microcomputer located in the spectrometer and configured to apply chemometrics to the system.

14. A method for detecting adulteration, moisture content, and origin of a food product, the method comprising:
receiving information related to a sample of the food product from a sensor for monitoring the food product and optionally testing environment synced through a wired and/or wireless communication network with a software application operating on a mobile computer device or on a computer device;
upon receiving the information, using a processor to determine an individual product profile and call up: (1) a plurality of predetermined individual chemometric measurements, which indicate adulterants, (2) a plurality of predetermined individual chemometric measurements, which indicate moisture content, (3) a plurality of predetermined individual chemometric measurements which indicate origin of the food product, and (4) at least one safety threshold, and a linked range of tolerance, related to the adulteration, moisture content, and origin of a food product, the predetermined individual chemometric measurements, threshold and range of tolerance having been previously uploaded by an individual or agency administering the test or an employee, contractor, or agent of the individual or agency
creating an individual risk profile by comparing the information received from the sensor against the predetermined individual chemometric measurements, which indicate adulterants, moisture content, or origin of the food product
comparing the individual risk profile to the at least one safety threshold, and a linked range of tolerance;
notifying the software application if the individual risk profile is above or below a specified safety threshold; and,
optionally, assigning additional resources when the security profile is above the specified threshold risk level.

15. The method of claim 14, further comprising at least two safety thresholds, and linked ranges of tolerances.

16. The method of claim 15, wherein at least one safety threshold and linked range of tolerance relates to: (a) adulterants, (b) moisture contents, (c) origin of the food product, or (d) a combination thereof.

17. The method of claim 16, wherein the system includes at least three safety thresholds and linked ranges of tolerance.

18. The method of claim 14, wherein the food product is honey.

19. The method of claim 14, wherein the sensor comprises:
a mid-infrared micro-Fourier transform infrared (FTIR) spectrometer configured to apply a spectrometric analysis to the sample;
an attenuated total reflectance (ATR) accessory located in the spectrometer and configured to receive the sample and operate on the sample by measuring the changes that occur in an internally reflected infrared beam when the beam comes in contact with the sample;
a microcomputer located in the spectrometer and configured to apply chemometrics to the system.

20. The method of claim 14, wherein the predetermined individual chemometric measurements are spectral images.
